(19) United States
(12) Patent Application Publication
(10) Pub. No.: US 2026/0335010 A1
(43) Pub. Date: Oct. 29, 2026

(54) SMART FEEDFORWARD FOR RF DELIVERY DURING RF PUNCTURE

(71) Applicant: Boston Scientific Scimed, Inc., Maple Grove, MN (US)

(72) Inventors: Steven Kinio, Pickering (CA); Aoting Chen, Toronto (CA); Prathima Sundaram, Brampton (CA); Deep Patel, Waterdown (CA)

(21) Appl. No.: 19/465,361

(22) Filed: Jan. 30, 2026

Related U.S. Application Data
(60) Provisional application No. 63/752,559, filed on Jan. 31, 2025.

(51) Int. Cl. A61B 18/16; A61B 18/00; A61B 18/14

(52) U.S. Cl. CPC A61B 18/16; A61B 18/1492; A61B 2018/00357; A61B 2018/00577; A61B 2018/00595; A61B 2018/167

(57) ABSTRACT

A radiofrequency (RF) generator for use in a medical system is disclosed. The radiofrequency generator includes a RF energy source and a controller. The RF energy source has an active connector and a return connector. The active connector is coupled to an active electrode of an RF puncture device and the return connector is coupled to a return electrode. The active electrode and return electrode are couple to a patient's body and form a patient circuit. The controller is coupled to the RF energy source. The controller determines an impedance of the patient circuit; determines a puncture signal based on the impedance of the patient circuit, and causes the RF energy source to apply the puncture signal to the patient circuit.

### CROSS REFERENCE TO RELATED APPLICATIONS

This application claims priority to U.S. Provisional Application No. 63/752,559 entitled “SMART FEEDFORWARD FOR RF DELIVERY DURING RF PUNCTURE,” filed Jan. 31, 2025, which is hereby incorporated by reference it its entirety.

### TECHNICAL FIELD

The present disclosure relates to medical devices and systems for use in percutaneous or interventional procedures including electrosurgery such as electrophysiology procedures. More specifically, this disclosure relates to electrosurgical units, such as radiofrequency (RF) generators, electrosurgical systems, and methods, that provide for impedance measurements.

### BACKGROUND

Catheters are often used to provide general access into a patient's body using minimally invasive techniques. In some examples, a catheter can be used to create a channel through a region of the body. One such example is a transseptal puncture in a cardiac procedure. The left atrium is a difficult cardiac chamber to access percutaneously. Although the left atrium can be reached via the left ventricle and mitral valve, the catheter is manipulated through two U-turns, which can be cumbersome. The transseptal puncture is a technique of creating a small surgical passage through the atrial septum, or wall in the heart between the left and right atrium, through which a catheter can be fed. The atrial septum is punctured and dilated via tools to create the passage. The transseptal puncture permits a direct route to the left atrium via the atrial septum and systematic venous system. Increasingly larger and complex medical devices can be passed into the left atrium. Historically, the technique was used exceptionally for mitral valvuloplasty and ablation in the left heart. Today, the increased interest in catheter ablation and its application in many other procedures has meant the transseptal puncture is a routine technique for interventional cardiologists and cardiac electrophysiologists.

Transseptal punctures can be performed with the aid of crossing devices having electrodes energized with a suitable power source such as an electrically coupled electrosurgical generator in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. Electrosurgical devices pass electrical energy through tissue between the electrodes to puncture tissue with plasma formed on the energized electrode. Tissue that contacts the plasma experiences a rapid vaporization of cellular fluid to produce a puncturing effect. Electrical energy from the electrosurgical generator can be applied to the electrodes either as a train of high frequency pulses or as a continuous signal typically in the radiofrequency (RF) range to perform the puncturing techniques.

### SUMMARY

In Example 1, a radiofrequency generator for use in a medical system, the radiofrequency generator comprising: a radiofrequency (RF) energy source having an active connector and a return connector, the active connector configured to be coupled to an active electrode of an RF puncture device and the return connector configured to be coupled to a return electrode, the active electrode and return electrode configured to couple to a patient body and form a patient circuit; and a controller coupled to the RF energy source, the controller configured to: determine an impedance of the patient circuit; determine a puncture signal based on the impedance of the patient circuit, and cause the RF energy source to apply the puncture signal to the patient circuit.

In Example 2, the radiofrequency generator of Example 1, wherein the controller is configured to cause the RF energy source to generate a test signal, wherein the test signal has a lower power than the puncture signal, and to determine the impedance of the patient circuit with the test signal.

In Example 3, the radiofrequency generator of any of Examples 1 and 2, wherein the determined impedance includes impedance of the RF puncture device.

In Example 4, the radiofrequency generator of any of Examples 1 and 2, wherein the determined impedance includes impedance between the active connector and the return connector.

In Example 5, the radiofrequency generator of any of Examples 1-4, wherein the determined impedance includes complex impedance having magnitude and phase angle information.

In Example 6, the radiofrequency generator of any of Examples 1 and 2, wherein the determined puncture signal is based on a feedforward control setting corresponding with the determined impedance.

In Example 7, the radiofrequency generator of Example 6, comprising an RF output circuit coupled to the controller, the RF output circuit to provide the RF energy source, wherein the controller is configured to apply the feedforward control setting to the RF output controller.

In Example 8, the radiofrequency generator of any of Examples 6 and 7, wherein the determined impedance corresponds with a voltage setting, wherein the controller is configured to apply the impedance and voltage setting to determine whether application of the puncture signal exceeds a threshold power setting.

In Example 9, the radiofrequency generator of any of Examples 6-8, wherein the controller is configured to adjust a nominal feedforward control setting based on the impedance to determine the feedforward control setting.

In Example 10, the radiofrequency generator of any of Examples 6-9, wherein the controller is configured to determine the feedforward control setting with a runtime calculation.

In Example 11, the radiofrequency generator of any of Examples 6-8, wherein the controller configured to determine the feedforward control setting with a lookup table.

In Example 12, the radiofrequency generator of any of Examples 6 and 7, wherein the controller is configured to determine the feedforward control setting and a voltage setting corresponding with the determined impedance with a lookup table.

In Example 13, the radiofrequency generator of Example 12, wherein the controller is configured to measure a voltage of the puncture signal in a steady state and update the lookup table.

In Example 14, the radiofrequency generator of Example 13 wherein the steady state is after the puncture signal has punctured tissue.

In Example 15, the radiofrequency generator of Example 1, wherein the RF puncture device is an electrosurgical transseptal guidewire.

In Example 16, a radiofrequency generator for use in a tissue puncture system for puncturing a tissue in a body, the radiofrequency generator comprising: a radiofrequency (RF) energy source having an active connector and a return connector, the active connector configured to be coupled to an active electrode of an RF puncture device and the return connector configured to be coupled to a return electrode, the active electrode and return electrode configured to couple to the body and form a patient circuit; and a controller coupled to the RF energy source, the controller configured to: determine an impedance of the patient circuit; determine a puncture signal based on the impedance of the patient circuit, and cause the RF energy source to apply the puncture signal to the patient circuit.

In Example 17, the radiofrequency generator of Example 16, wherein the controller is configured to cause the RF energy source to generate a test signal, wherein the test signal has a lower power than the puncture signal, and to determine the impedance of the patient circuit with the test signal.

In Example 18, the radiofrequency generator of Example 16, wherein the determined impedance includes impedance of the RF puncture device.

In Example 19, the radiofrequency generator of Example 16, wherein the determined impedance includes impedance between the active connector and the return connector.

In Example 20, the radiofrequency generator of Example 16, wherein the determined impedance includes complex impedance having magnitude and phase angle information.

In Example 21, the radiofrequency generator of Example 16, wherein the determined puncture signal is based on a feedforward control setting corresponding with the determined impedance.

In Example 22, the radiofrequency generator of Example 21, comprising an RF output circuit coupled to the controller, the RF output circuit to provide the RF energy source, wherein the controller is configured to apply the feedforward control setting to the RF output controller.

In Example 23, the radiofrequency generator of Example 21, wherein the determined impedance corresponds with a voltage setting, wherein the controller is configured to apply the impedance and voltage setting to determine whether application of the puncture signal exceeds a threshold power setting.

In Example 24, the radiofrequency generator of any of Example 21, wherein the controller is configured to adjust a nominal feedforward control setting based on the impedance to determine the feedforward control setting.

In Example 25, the radiofrequency generator of Example 21, wherein the controller is configured to determine the feedforward control setting with a runtime calculation.

In Example 26, the radiofrequency generator of Example 21, wherein the controller configured to determine the feedforward control setting with a lookup table.

In Example 27, the radiofrequency generator of Example 21, wherein the controller is configured to determine the feedforward control setting and a voltage setting corresponding with the determined impedance with a lookup table.

In Example 28, the radiofrequency generator of Example 27, wherein the controller is configured to measure a voltage of the puncture signal in a steady state and update the lookup table.

In Example 29, the radiofrequency generator of Example 28 wherein the steady state is after the puncture signal has punctured tissue.

In Example 30, the radiofrequency generator of Example 16, wherein the RF puncture device is an electrosurgical transseptal guidewire.

In Example 31, a radiofrequency generator for use in a tissue puncture system for puncturing a tissue in a body, the radiofrequency generator comprising: a radiofrequency (RF) energy source having an active connector and a return connector, the active connector configured to be coupled to an active electrode of an RF puncture device and the return connector configured to be coupled to a return electrode, the active electrode and return electrode configured to couple to the body and form a patient circuit; and a controller coupled to the RF energy source, the controller configured to: cause the RF energy source to generate a test signal; determine an impedance of the patient circuit from the test signal; determine a puncture signal based on the impedance of the patient circuit, wherein the puncture signal has a higher power than the test signal, wherein the determined puncture signal is based on a feedforward control setting corresponding with the determined impedance; and cause the RF energy source to apply the puncture signal to the patient circuit.

In Example 32, the radiofrequency generator of Example 31, comprising an RF output circuit coupled to the controller, the RF output circuit to provide the RF energy source, wherein the controller is configured to apply the feedforward control setting to the RF output controller.

In Example 33, the radiofrequency generator of Example 32, wherein the controller is configured to determine the feedforward control setting and a voltage setting corresponding with the determined impedance with a lookup table, and wherein the controller is configured to measure a voltage of the puncture signal in a steady state and update the lookup table.

In Example 34, a method for use in a radiofrequency (RF) generator in a tissue puncture system, the RF generator including an RF energy source having an active connector and a return connector, the active connector configured to be coupled to an active electrode of an RF puncture device and the return connector configured to be coupled to a return electrode, the active electrode and return electrode configured to couple to a patient body and form a patient circuit, the method comprising: determining an impedance of the patient circuit; determining a puncture signal based on the impedance of the patient circuit, and causing the RF energy source to apply the puncture signal to the patient circuit.

In Example 35, the method of Example 34, comprising generating a test signal with the RF energy source, wherein the test signal has a lower power than the puncture signal; and determining the impedance of the patient circuit with the test signal.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary electrosurgical system for performing an electrosurgical puncture, such as a transseptal puncture.

FIG. 2 is a block diagram illustrating an embodiment of an electrosurgical generator configured for use with the electrosurgical system of FIG. 1.

FIG. 3 is a block diagram illustrating an embodiment of a method of applying an electrical puncture signal with a determined impedance using the electrosurgical generator of FIG. 2 in the electrosurgical system of FIG. 1.

FIG. 4 is a graph illustrating plots of electrical current with respect to time and voltage with respect to time in an example application of the puncture signal with the electrosurgical generator of FIG. 2.

FIG. 5 is a block diagram illustrating an embodiment of a method of a feature of the method of FIG. 3.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Tissue punctures via radiofrequency (RF) energy are associated with thermal energy transfer between a puncture electrode and target tissue. Thermal energy transfer factors used in evaluating effective tissue puncture include temperature at the tissue-electrode interface and thermal ramp rates, or how quickly the interface can get to the desired temperature. The desired temperature of the interface for a tissue crossing preferably achieves tissue vaporization in which water inside of the tissue cells flashes to steam and bursts the cells. Tissue fulguration occurs if too much thermal energy is delivered to the interface, and fulguration can include aggressive heating of blood and damage to tissue outside of the target region. Too high of a thermal ramp rate can cause the interface to overshoot its target temperature, which also leads to fulguration. On the other hand, tissue desiccation occurs if not enough thermal energy is delivered to the interface, and desiccation results in dried tissue that forms a scar. Too low of a thermal ramp rate can cause the interface to spend too much time in the tissue desiccation temperature region, which also leads to desiccation.

Electrosurgical generators include circuits to provide RF energy sources for tissue punctures. To achieve rapid heating to target temperatures, electrosurgical generators can leverage voltage-feedback control to target voltage that are known to produce desirable temperatures at the puncture electrodes. A standard Proportional-Integral-Derivative (PID) controller, which applies a closed-loop feedback mechanism, ramps voltages too slowly to achieve a desirable puncture, so an electrosurgical generator can apply a feedforward approach with an often preprogrammed, nominal control setting to achieve rapid heating to target temperatures. High voltage output circuits used in electrosurgical generators, however, are well tuned to delivery energy to specific loads, and load changes can cause high voltage circuitry to behave differently than expected. For example, a first control setting (such as 500) will produce 100 V for a 1 kΩ load, but the first setting might produce 140 V for a 2 kΩ load. In a clinical setting, every patient and operating theatre presents a particular electrical load to the electrosurgical generator. Further, as high voltage output circuits “burn-in” from use, some characteristics will drift, which can also affect load dependence. This load dependence is why closed-loop feedback, such as via a PID controller, is applied during energy delivery. But feedback strategies to compensate for load dependence are not beneficial for determining feedforward settings. Incorrect feedforward settings can result in an overvoltage or undervoltage condition that can affect settling over time or other unwanted effects during a medical procedure. The disclosure is related to an electrosurgical generator, electrosurgical systems, and methods to determine feedforward settings prior to the delivery of RF energy to account for load dependence.

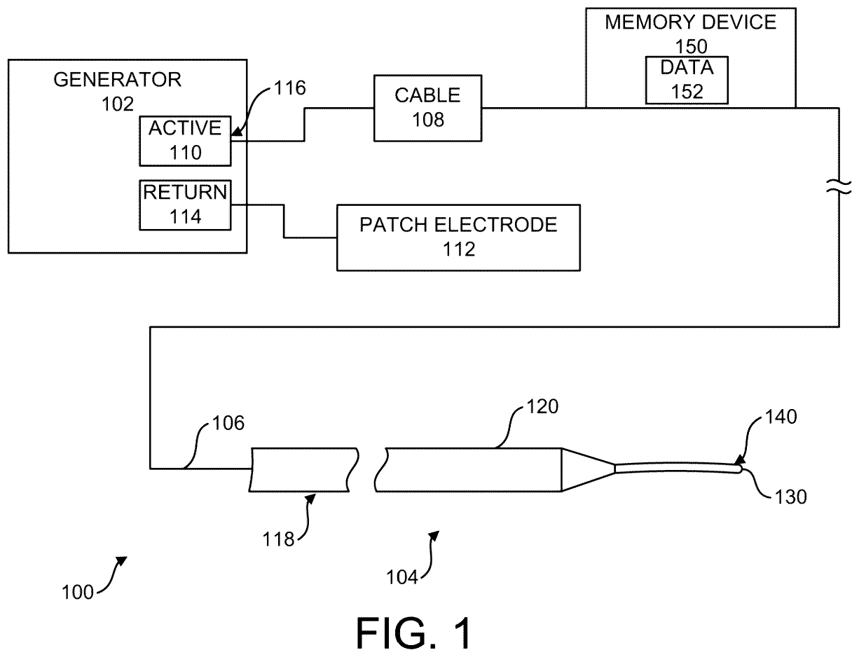

FIG. 1 illustrates an embodiment of an electrosurgical system 100 to facilitate vascular access to a heart and provide catheter positioning within cardiac anatomy. The embodiment of the medical system 100 includes an electrosurgical unit, such as an electrosurgical generator 102 and an electrosurgical device, such as an electrosurgical puncture device 104. In one embodiment, the electrosurgical puncture device 104 includes an electrosurgical transseptal guidewire 106. In the illustration, the electrosurgical transseptal guidewire 106 is electrically coupled to the electrosurgical generator 102 via cable 108, such as to an active connector 110 on the electrosurgical generator 102. The electrosurgical generator 102 is configured to provide a source of energy, such as RF energy to the electrosurgical transseptal guidewire 106 via the cable 108. In some embodiments, the system 100 includes a ground pad electrode, or indifferent (dispersive) patch electrode 112 electrically coupled to the electrosurgical generator 102, such as to a return connector 114 on the electrosurgical generator 102, for use with the electrosurgical transseptal guidewire 106 in a monopolar configuration. In some embodiments, the electrosurgical transseptal guidewire is implemented in a bipolar configuration using a pair of electrodes on the guidewire and without a separate patch electrode. The active connector 110 and return connector 114 are included as a set of RF receptacles 116 on the generator 102.

The electrosurgical generator 102 is configured to provide the source of RF energy to the electrosurgical transseptal guidewire 106 for a puncture operation with the electrosurgical device 104. In some embodiments, the electrosurgical generator 102 includes an interface including a set of user accessible controls, device connectors such as receptacles 116, and an output device such as a display device, speakers, and lights. During a monopolar puncture operation of electrosurgical generator 102, a first electrode, often referred to as the active electrode, is provided with the electrosurgical device 104 in general and with the transseptal guidewire 106 in the illustration while a second electrode, such as the patch electrode 112, is typically located on the back, buttocks, upper leg, or other suitable anatomical location of the patient during surgery. In such a configuration, the patch electrode 112 is often referred to as a patient return electrode. An electrical circuit of RF energy is formed between the active electrode and the patch electrode 110 through the patient as a patient circuit, and an RF puncture signal through the patient circuit is used to puncture tissue at the active electrode. For example, RF energy for a puncture function in a monopolar mode may be provided at a relatively low voltage and a continuous current (100% on, or 100% duty cycle). At a power setting of 50 Watts for puncturing (although instantaneous power may be higher), voltage can range from approximately 164 to 400 volts root mean square (RMS). The electrosurgical generator 102 can include a plurality of functions and provide a programmed and custom settings via an interface and be couplable to a suite of electrosurgical devices in addition to the transeptal guidewire 106. In one example, the electrosurgical generator 102 provides RF energy to the active electrode as an alternating current having a frequency in the range of 100 kHz to 10 MHz. Typically, this energy is applied in the form of a continuous sinusoidal puncture signal. In some embodiments, the energy is applied in bursts of pulses. The individual pulses in each burst of a pulsed puncture signal typically each have a duration of 300 milliseconds with an interval between pulses of 700 milliseconds but can vary such as based on parameters of the connected electrosurgical device 104. The actual pulses are often sinusoidal or square waves and bi-phasic, that is alternating positive and negative amplitudes.

The illustrated electrosurgical puncture device 106 includes the electrosurgical transseptal guidewire 106 and a delivery component 118. While embodiments of the disclosure are described with reference to punctures in tissue with a transseptal guidewire for illustration, the features of the disclosure can be used with other electrosurgical devices. The delivery component 118 includes an elongated shaft 120 having a shaft distal tip and defines a longitudinally extending axial shaft lumen. The electrosurgical transseptal guidewire 106 is adapted to be disposed within the shaft lumen and electrically coupled to the RF energy source at the active connector 110. In some embodiments, the delivery component 118 can include an elongate sheath, and the electrosurgical transseptal guidewire 106 is disposed within the sheath. In another embodiment, the delivery component 118 can include a dilator/sheath assembly, and the electrosurgical transseptal guidewire 106 is disposed within the dilator/sheath assembly. For instance, the elongated shaft 120 includes a distal tapered portion 122 with an enlargement of cross-sectional area with respect to the shaft distal tip. As the distal tapered portion 122 is passed through an aperture from the shaft distal tip, the enlargement of cross-sectional area dilates the aperture. The dilator can be configured as a straight dilator, as illustrated, or a curved dilator. The elongated shaft 120 can be made from various materials including insulative materials such as high-density polyethylene (HDPE). The shaft 120 and distal tapered portion 122 can include various materials such as metal hypotubes as well.

The electrosurgical transseptal guidewire 106 includes a puncture wire shaft with a puncture wire proximal portion and a puncture wire distal portion having a puncture wire distal tip 130. The puncture wire distal tip 130 includes a puncture electrode 140 adapted to deliver the RF energy. The puncture electrode 140 is configured as the active electrode. The puncture wire proximal portion includes an end connector configured to electrically couple to cable 108 and receive an RF signal from the electrosurgical generator 102. In one example, the electrosurgical transeptal guidewire 106 can be coupled to and uncoupled from the cable 108 depending on whether the electrosurgical transeptal guidewire 106 is used as an electrosurgical puncture device or as an exchange rail, for instance. The transseptal guidewire 106 is configured to conduct the RF signal from the proximal portion along the puncture wire shaft to the electrode 140. In some embodiments, the puncture wire shaft is constructed from an electrically conductive material having an insulative outer coating. In some embodiments, the electrically conductive material is a flexible, shape memory material such as a nickel titanium alloy or nitinol. The exposed electrode 140 is configured to apply the RF energy, such as to puncture tissue.

In the illustrated example, the electrosurgical transseptal guidewire 108 is configured as a multifunction conductive guidewire. For instance, the transseptal guidewire 108 can be used, without exchanges, as a guidewire, a transseptal puncture device, and as an exchange rail for delivering therapy sheaths. Such embodiments provide efficiencies to medical procedures as the transseptal guidewire 106 performs multiple functions and reduces the amount of device exchanges in the medical procedure. The distal tip 130 of the transseptal guidewire 106 is extendable from the distal end of the delivery component 118 such that the delivery component 118 is retractable from the patient over the guidewire 106 while the guidewire distal tip 130 is disposed within the heart. The transseptal guidewire 106 is sufficiently thin and flexible to access the various chambers of the heart. The electrode 140 on the transseptal guidewire 106 is operable to deliver RF energy to puncture the atrial septum from the right atrium, and the distal portion of the transseptal guidewire can be advanced through an aperture in the septum formed by the puncture. Once advanced through the aperture and sufficiently extended from within the delivery component 118, the distal portion of the transseptal guidewire 106 is biased to form a coil for anchoring the distal end beyond the aperture. The delivery component 118 is retractable from the patient over the anchored transseptal guidewire 106. The transseptal guidewire 106 can also support the installation of therapy devices to a therapy location in the patient's heart, such as tubular members or other catheters and for advancing other devices within the heart.

In some embodiments, the electrosurgical generator 102 is automatically programmed for operation based on the particular configuration of the electrosurgical device 104. The transseptal guidewire 106 of the illustrated embodiment includes a memory device 150 (non-transitory memory) storing a set of parameters 152 associated with the design and configuration of the transseptal guidewire 106. The memory device 150 is coupled to the electrosurgical generator 102 via the receptacles 116, such as a serial interface, and the electrosurgical generator 102 is configured to read the parameters 152 to program the controls to be suited for the associated transseptal guidewire 106. The memory device 150 can store the parameters 152 in various memory segments having lookup tables or other data structures to provide data to be loaded into a memory device in the electrosurgical generator 102 and read by a controller of the electrosurgical generator to affect operation. Example parameters 152 can include model number of the transseptal guidewire 106, acceptable voltages or power levels for use with the transseptal device 106, whether the transseptal device 106 is configured for single use or multiple uses, impedance of the transseptal device 106, as well as other parameters. In some embodiments, the electrosurgical generator 102 can be programmed to write to memory segments on the memory device 150 as well as to read the memory device 150.

In an anticipated use of the system 100, the electrosurgical device 106 is coupled to the electrosurgical generator 102, and if the electrosurgical device 104 is to be configured in a monopolar mode, the patch electrode 112 is coupled to the patient. The electrosurgical generator 102 can be set to a puncture mode, such as an energy output of approximately 10 watts. In some examples, femoral access is obtained via a conventional percutaneous needle, and the transseptal guidewire 106 is inserted into the vasculature and advanced to the superior vena cava. The distal tip of the delivery component 118 is advanced over the proximal portion of the transseptal guidewire 106, to the superior vena cava. Under visualization, the distal tapered portion 122 is moved from the superior vena cava to the right atrial septum and then to the fossa ovalis of the heart. Once the delivery component distal tip is confirmed at the fossa ovalis, the electrode 140 of the transseptal guidewire 108 is advanced from the distal tip of the delivery component 118. In one example, the exposed puncture electrode 140 of the transseptal guidewire 108 is extended a few millimeters from the distal tip of the delivery component 118 to tent the heart tissue, and the transseptal guidewire 106 can be locked in position with respect to the delivery component 118. Forward pressure is applied to the electrosurgical device 104 and the transseptal guidewire 106 is actuated to apply the RF energy to the electrode 140 and puncture the fossa ovalis. The RF energy punctures the fossa ovalis and creates the aperture. The transseptal guidewire 108 is unlocked from the delivery component 116, and the transseptal guidewire 106 is extended through the aperture. In the embodiment of the delivery component 116 configured as the dilator/sheath assembly, the distal tapered portion 122 of a dilator is advanced into the puncture site to expand the aperture in the septum. The delivery component 118 can be retracted from the patient over the transseptal guidewire 106, and transseptal guidewire 106 can provide support for the installation of tubular members or other catheters and for advancing other devices within the heart.

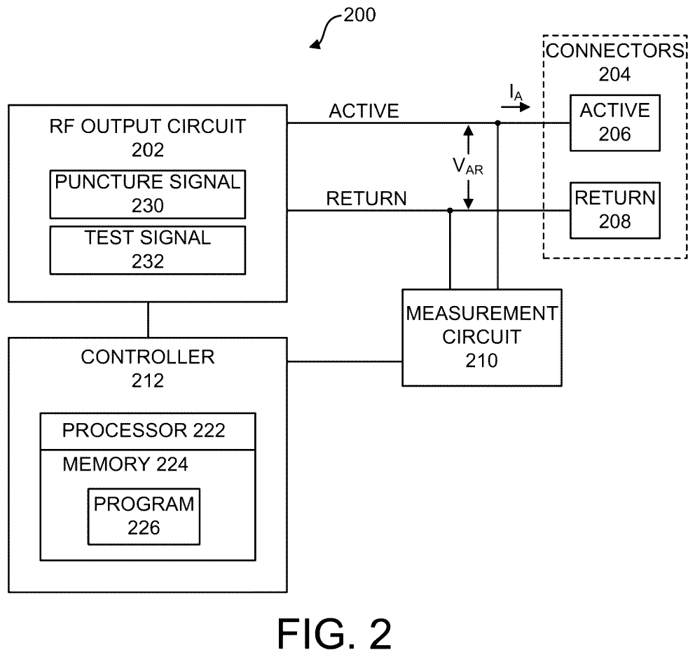

FIG. 2 illustrates an embodiment of electrosurgical unit, such as an electrosurgical generator 200, which can correspond with electrosurgical generator 102 in system 100. The electrosurgical unit or RF generator is described with reference to transseptal punctures in this disclosure for illustration only. But the principles of the electrosurgical generator of this disclosure are not limited to transseptal punctures. For instance, the RF generator 200 can be used in other medical systems including systems for ablation and electrocautery. The electrosurgical generator 200 is configured to generate an RF puncture 230 signal based on a nominal feedforward control setting. In embodiments, the electrosurgical generator 200 is also configured to determine an electrical load coupled to and including an associated electrosurgical device such as the transseptal guide wire 106 based on impedance measurements taken from a relatively low power, high frequency test signal 232. The electrosurgical generator 200 can measure an impedance of the test signal 232, such as a complex impedance and phase angle, and adjust the nominal feedforward control setting for the RF puncture signal 230 such as via applying a load dependent correction factor to the nominal feedforward control setting. In some embodiments, the impedance measurement taken from the test signal 232 or the RF puncture signal 230 can be used to adjust the load dependent correction factor and can compensate for changes in hardware as circuit age over time, which might result in additional variations of how voltage outputs respond to control inputs.

The electrosurgical generator 200 includes an RF output circuit 202, a plurality of device connectors 204 including an active connector 206 and a return connector 208, a measurement circuit 210, and a controller 212. In one embodiment, the RF output circuit 202 is configured to generate an RF puncture signal 230 and a relatively lower power RF test signal 232. The RF output circuit 202 can generate an RF puncture signal 230 and the RF test signal 232 at the same or different frequencies. The RF output circuit 202 generates the puncture signal 230 based on feedforward settings determined from the controller 212 and, in some embodiments, applies closed-loop feedback via a PID controller during delivery of the puncture signal 230. In some embodiments, the controller 212 determines the feedforward setting from an application of the test signal 232. The RF output circuit 202 can include a power supply to provide a direct current (DC) supply voltage signal to an RF voltage converter that converts the DC signal to an alternating current (AC) signal as an RF voltage. The RF voltage is provided to a waveform generator of the RF output circuit 202, which is configured to generate a plurality of voltages, waveforms having various duty cycles, peak voltages, crest factors, frequencies, and other suitable parameters. The RF output circuit provides the RF test signal 232 and the puncture signal 230 to the active connector 206.

The device connectors 204 can be configured to include receptacles located on a housing of the RF generator 200 that can be mechanically coupled to electrosurgical devices. The device connectors 204 are configured to electrically couple the electrosurgical generator 200 to various electrosurgical devices. In some embodiments, the device connectors 204 correspond with the RF receptacles 116 of FIG. 1. In some embodiments, the active connector 206 is suitable for electrically coupling to cable 108, which can be electrically coupled to the transseptal guidewire 106. The return connector 208 is suitable for electrically coupling to the ground pad electrode 112 when an electrosurgical device is operated in a monopolar mode (or to a return electrode on the electrosurgical device when operated in a bipolar mode). During a medical procedure, the electrode 140 of the transseptal guidewire 106 and the return electrode, such as the ground pad electrode 112, are coupled to the patient, such heart tissue and the torso, respectively, to form a patient circuit with the RF output circuit 202 for the puncture signal 230 and the test signal 232.

The measurement circuit 210 is electrically coupled to the device connectors 204 and is configured to determine current and voltage measurements or impedance measurements from an excitation waveform resulting from the test signal 232 and the puncture signal 230 generated by the RF output circuit 202 in the patient circuit and present the current and voltage or impedance measurements to the controller 212. In some embodiments, the measurement circuit 210 includes circuit elements that measure aspects of impedance, such as electrical current and voltage, from a test signal 232 prior to the delivery of an RF puncture signal 230 and from the puncture signal 230. The measurement circuit 210 can include circuit elements or paths electrically coupled to the RF output circuit 202 or at least some of the output connectors 204 including the active connector 206 and return connector 208 and is configured to provide a signal representative of the active and return voltages and active current. The circuit elements can include current probes to measure currents of interest. In one embodiment, the measurement circuit 210 includes an analog to digital converter coupled to the circuit elements and to the controller 212 to provide digital signals to the controller 212.

The controller 212 is coupled to the output circuit 202 and is configured to control, such as to adjust settings and cause operation of, the output circuit 202. The controller 212 in embodiments includes a processor 222 operably connected to a memory device 224 (non-transitory memory). The memory device 224 can store processor executable instructions configured to control the processor, such as a program 226. Examples of a memory device 224 can include a non-volatile memory device such as a read only memory (ROM), electronically programmable read only memory (EPROM), flash memory, non-volatile random-access memory (NRAM) or other memory device, and a volatile memory device such as random-access memory (RAM) or other memory device. In some embodiments, memory device 224 includes a non-volatile component to store a nominal feedforward control setting to be adjusted by the program 226. In some embodiments, the memory device 224 includes a non-volatile component for storing tables or other data structures used with the program 226. The processor 222 includes an output port that allows the processor 222 to control the output of the controller 212 according to a programmed scheme.

In other embodiments, the functionalities of controller 212 are at least partially implemented in the form of electronic circuitry. Examples of electronic circuitry include integrated circuits including ASICs and programmable logic devices, such as field programmable gate arrays. A field programmable gate array is a type of integrated circuit that can be programmed or reprogrammed after manufacture and include programable logic blocks and interconnects that are configured to perform various digital functions. The logic blocks can be configured to perform combinational functions or as logic gates. Logic blocks can also include memory elements, such as flip-flops or more complete memory devices including volatile and non-volatile memory aspects that can include look up tables. Functions can be defined via a hardware description language in an electronic design automation tool to create a binary file to configure the electronic circuitry. Those skilled in the art recognize that descriptions of methods, processes, of this disclosure illustrated with the processor 222, memory 224, and program 226 can be implemented in such electronic circuitry.

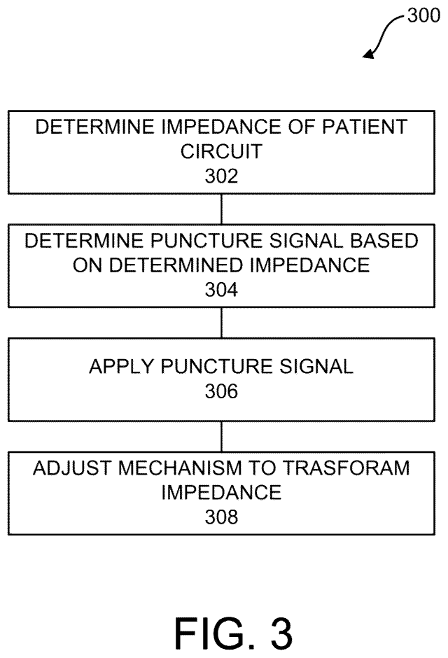

FIG. 3 illustrates an embodiment of a method 300 to perform an RF puncture delivery based on feedforward voltage control setting determined from impedance in the patient circuit. In embodiments, the method 300 is implemented with the electrosurgical generator 200, such as via a set of executable instructions in program 226 of the controller 212. The electrosurgical generator 200 provides the RF energy source and the active connector 206 is configured to be coupled to an active electrode of an RF puncture device and the return connector 208 is configured to be coupled to a return electrode. The active electrode and return electrode are configured to be coupled to the body, such as the heart tissue and torso, respectively, and form a patient circuit and an electrical load defining an impedance coupled between the active and return connectors 206, 208. Using method 300, an impedance of the patient circuit is determined, at 302. A puncture signal is determined based on the impedance of the patient circuit at 304. A number of mechanisms to transform the determined impedance of the patient circuit into the puncture signal are available, such as a runtime calculation through parameters or mapping. An RF energy source applies the puncture signal to the patient circuit to puncture tissue at 306. In some embodiments, the mechanism to transform the determined impedance of the patient circuit into the puncture signal is adjusted, such as updated at 308.

In one embodiment, the impedance of the patient circuit is measured at 302 via the electrosurgical generator 200. Impedance is the opposition to an AC signal presented by the combined effect of resistance and reactance in the circuit. In embodiments, impedance is measured via the same circuit path as used to deliver a high voltage puncture signal but with a test signal providing a low voltage excitation. The controller 212 measures impedance of the test signal via the measurement circuit 210. In one embodiment, the controller 212 configures the RF output circuit 202 to generate and deliver the test signal to the active connector 206 at a selected frequency. In one embodiment, a low power, high frequency test signal is provided at signal levels that are high enough to perform impedance measurements with the electrosurgical generator 102, such as to make an electrical path with a current from the puncture electrode 140 through the patient to the patch electrode 112 and return connector 208, at a frequency high enough to avoid stimulation of cardiac tissue during the measurement (over about 100 kHz), and of a power low enough to avoid an adverse effect to the patient from the test signal. In one embodiment, the test signal is about 300 millivolts peak-to-peak at about 200 kHz. Thus, the impedance measurements can be taken at a time different than the time of the RF puncture, such as prior to application of a puncture signal. The RF output circuit 202, in one embodiment, continuously provides the test signal and interrupts the test signal when a high power, high frequency RF puncture signal is applied to the active connector 206 to puncture tissue. In another embodiment, the RF output circuit 202 provides the test signal as a discrete pulse, or train of pulses, for a short period of time according to a programmed scheme.

The measurement circuit 210 receives the resultant test signal and, in one embodiment, converts the analog signal to a digital signal for the controller 212 to process. The measurement circuit 210 is electrically coupled to the active and return connectors 206, 208 and determines electrical current waveforms and voltage waveforms of the resultant test signal, such as current at the active connector 206, Iₐ, and voltage across the active connector 206 and return connector 208, Vₐᵣ, performs an analog to digital conversion of the determination, and provides the digitized measurement to the controller 212. Impedance of the patient circuit Zₚₐₜᵢₑₙₜ is determined via (Vₐᵣ)/(Iₐ). In one embodiment, the measurement circuit 210 determines both the voltage Vₐᵣ and current Iₐ to determine both the magnitude and phase angle for the complex impedance of the electrical load coupled to the active and return connectors 206, 208. In one embodiment, the controller 212 determines complex impedance measurements via a Fourier transform technique on the voltage Vₐᵣ and current Iₐ data of the measured test signal. In other embodiments, the complex impedance is determined via under-sampling/over-sampling the voltage Vₐᵣ and current Iₐ to create a harmonic equivalent of the resultant waveform of the test signal and performing a sum-of-least-squares calculation to measure root-mean-square equivalents of the waveform, which is used to calculate impedance. In still another embodiment, hardware is applied to convert the voltage Vₐᵣ and current Iₐ to equivalent direct current signals, which is used to extract phase information. Other mechanisms and processes can be applied to determine the complex impedance.

The puncture signal is determined based on the impedance of the patient circuit at 304. In embodiments, a feedforward control setting is determined based on the impedance of the patient circuit, and the feedforward control setting is applied to the RF output circuit 202 to generate the puncture signal. In one embodiment, a nominal feedforward control setting is stored in memory 224 and is adjusted via the impedance of the patient circuit to obtain an output feedforward control setting that is applied to the RF output circuit 202. By way of example, an electrosurgical generator 200 uses a control mechanism or circuit to set to the voltage level delivered to the patient. For instance, the electrosurgical generator 200 can use a controllable digital-to-analog (DAC) converter to set the feedforward volage (V_{FF}) delivered to the device connectors 204 from the RF output circuit 202. In some embodiments, the DAC receives a signal based on a control setting from the controller of a generator and applies the control setting to adjust an output voltage. In a typical example, the control setting applied to the DAC, or DAC_{SETTING}, to create an output voltage from a selected feedforward voltage (V_{FF}) can be determined from a linear relation such as:

DAC_{SETTING},=V_{FF}*m+b

In the electrosurgical generator 200, the control setting applied to the DAC are determined from:

DAC_{SETTING},=(V_{FF}*f(Zₚₐₜᵢₑₙₜ))*m+(b*g(Zₚₐₜᵢₑₙₜ))

in which f(Zₚₐₜᵢₑₙₜ) and g(Zₚₐₜᵢₑₙₜ) are correction functions that leverage the determined impedance of the patient circuit Zₚₐₜᵢₑₙₜ at 302. The feedforward voltage V_{FF} in one embodiment is the nominal feedforward voltage value, such as the feedforward voltage expected to be obtained via a nominal feedforward control setting. In one embodiment, the control setting to apply a feedforward voltage is calculated from a formula based on the measured impedance of the associated patient circuit.

In another embodiment, the control setting to apply a feedforward voltage is determined via a memory structure such as an array or lookup table (LUT), such as a control setting LUT, stored in memory 224 and configured to be accessed by the processor 224 and program 226. The use of the LUT in process 300 can include advantages such as a faster determination of the control setting over a runtime computation using processing resources. The control setting LUT can be a multidimensional LUT constructed in which a set of impedances at a given frequency of the test signal is mapped to a corresponding control setting for use with the controller. In one embodiment, each impedance includes a magnitude and phase angle. The control setting LUT can be populated with many impedances over a wide range of impedances as nodes, and each node is mapped to a corresponding control setting, such as DAC_{SETTING} in embodiments that adjust the DAC to apply the feedforward control. In one embodiment, a determined impedance of the patient circuit is input into the LUT, such as the patient circuit impedance Zₚₐₜᵢₑₙₜ measured at 302, and a control setting is output, such as DAC_{SETTING}, which is applied to the RF output setting to generate the puncture signal. The determined impedance either matches a node value in the table, in which case the corresponding control setting is output, or interpolation is applied to output the corresponding control setting.

In one embodiment, the feedforward control setting can be used to determine the puncture signal target voltage, which is to be achieved with the ramp up of an applied puncture signal at 306. In some examples, the puncture signal target voltage of the electrosurgical generator can be stored in the LUT and associated with the impedance of the patient circuit and corresponding control setting. In some embodiments, the input of a measured impedance of the patient circuit Zₚₐₜᵢₑₙₜ returns the corresponding control setting as well as a corresponding puncture signal target voltage V_{target}, which (in one example) is the generally the expected voltage of the puncture signal reached at a set point after the ramp up. In some embodiments, the puncture signal target voltage V_{target}, can be applied to determine whether the electrosurgical generator 200 can produce enough electrical power at the setpoint Pₛₑₜₚₒᵢₙₜ. Once the control setting and puncture signal target voltage V_{target} have been determined, such as output from the LUT, the electrical power at the setpoint Pₛₑₜₚₒᵢₙₜ can be determined via the equation:

V_{target}=sqrt(Pₛₑₜₚₒᵢₙₜ* Zₛₑₜₚₒᵢₙₜ

in which sqrt( ) is the square root function. If the determined electrical power at the setpoint Pₛₑₜₚₒᵢₙₜ crosses a threshold, such as exceeds a threshold power level of the electrosurgical generator associated with a safety threshold, the method 300 can produce an alert and prevent the puncture signal from being applied at 306. If the determined electrical power at the setpoint Pₛₑₜₚₒᵢₙₜ does not cross the threshold, such as does not exceed safety threshold power level of the electrosurgical generator, the method 300 can allow the application of the puncture signal to proceed at 306.

In some embodiments, the mechanism to transform the determined impedance of the patient circuit into the puncture signal is adjusted at 308. In embodiments, the parameters of a runtime calculation or the control setting LUT is updated so as to affect future puncture signal deliveries. In some examples, patients, the operating theatre, or circuit burn in can generate variances in the high voltage output that are not accounted for during manufacturing of the electrosurgical generator. By adjusting the mechanism to transform the impedance into the puncture signal at 308, an electrosurgical generator can adapt to variances due to specific circumstances.

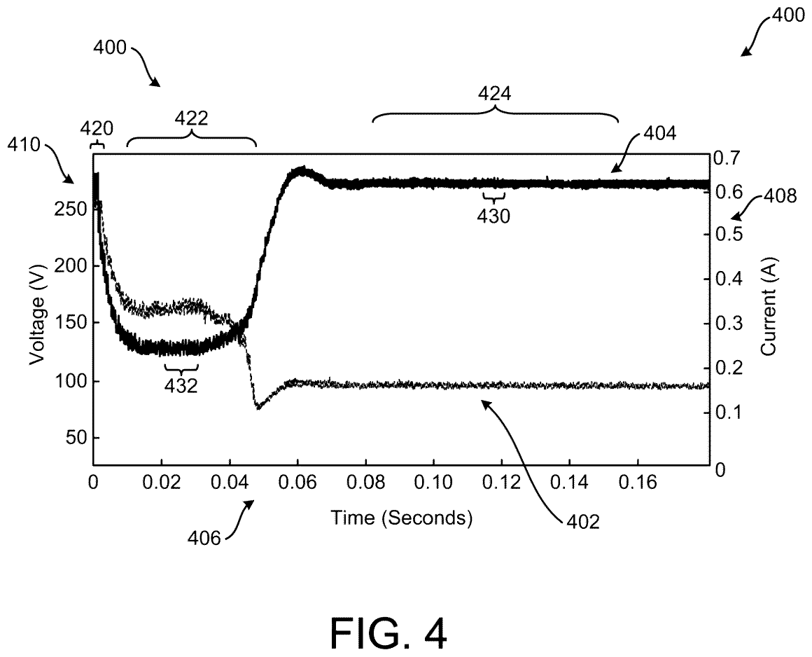

FIG. 4 illustrates a graph 400 including a plot of electrical current magnitude 402 and voltage magnitude 404 of a puncture signal with respect to time as measured by a measurement circuit 210 of an electrosurgical generator 200 during a tissue puncture procedure with a transseptal guidewire 106. A timeline, or time since activation of the puncture signal in the puncture procedure in seconds is 406 is presented along an x-axis. Current in amperes (A) root-mean-square (RMS) is presented along a right-side y-axis 408, and voltage in volts (V) RMS is presented along the left-side y-axis 410. Time 406 ranges from zero seconds to approximately 0.18 seconds on the graph 400. Puncture signal current 408 ranges from zero amperes to 0.7 amperes on the graph 400. Puncture signal voltage ranges from approximately 25 volts RMS to approximately 300 volts RMS on the graph 400. For the purposes of illustration, the puncture signal delivery is generally separated into three stages with respect to time, a ramp up stage 420, a puncture stage 422, and a final steady state stage 424. During the final steady stage 422, the voltage magnitude 404 remains in a relatively narrow voltage range over time or first steady state delivery range 430 of the puncture signal, in which variances in voltage magnitude of the puncture signal can be due to noise or artifacts. A second steady stage delivery range 432 can also occur, often for a relatively briefer period of time, during the puncture stage 422. As the voltage magnitude 404 remains in the relatively narrow voltage ranges over time during the first and second steady state delivery ranges 430, 432 with respect to time, the PID of a puncture signal feedback mechanism in the RF output detects a steady state delivery of the puncture signal. Information regarding the steady state delivery can be provided to the controller, such as the voltage magnitudes during the first and second steady state delivery ranges 430, 432. Such information can be applied in making the adjustments at 308.

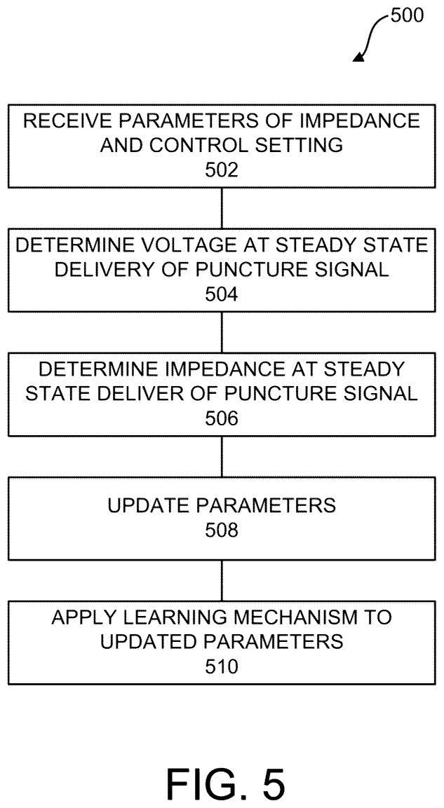

FIG. 5 illustrates an embodiment of a method 500 to adjust the transformation of the determined impedance of the patient circuit into the puncture signal. In embodiments, the method 500 is implemented with the electrosurgical generator 200, such as via a set of executable instructions in program 226 of the controller 212. In some embodiments, method 500 is performed as 308 in method 300. For example, the impedance of the patient circuit is determined at 302 and the puncture signal based on the impedance is determined at 304. In one embodiment, impedance of the patient circuit and the corresponding control setting are received at 502. In one embodiment, the impedance of the patient circuit, such as the impedance magnitude and phase angle, and the corresponding control setting are included as parameters of the node of an LUT. In some embodiments, the method receives other parameters of node such as a node identifier and date of last update associated with the node. While the puncture signal is applied using the associated control setting at 306, a voltage magnitude at an identified steady state delivery range is determined at 504. For example, the controller 212 detects a steady state delivery of the puncture signal, such as based on operation of the PID, which results in a range of the voltage magnitude at a relatively steady state for a period of time. An example of the range of the voltage magnitude at a relatively steady state include the first steady state delivery range 430 of FIG. 4, or, in some circumstances, another example of the range of the voltage magnitude at a relatively steady state the second steady state delivery range 432 of FIG. 4. During the steady state delivery range, the steady state voltage V_{SS} and steady state current I_{SS} are measured, such as the RMS V_{SS} and RMS I_{SS}, to determine a steady state impedance of the puncture signal Z_{SS} at 506. The measured steady state voltage V_{SS}, the determined impedance of the puncture signal Z_{SS}, and the associated control setting (received at 502) are written to an adjusted or updated node in the LUT at 508. The updated LUT can be subjected to a learning mechanism, such as an artificial intelligence application, to determine a new LUT or a new subset of data within a LUT used to provide control settings at 510.

In one embodiment of applying a learning mechanism to provide control settings, the learning mechanism can determine whether the control settings are outside of a tolerable range and whether new control setting are recommended. The learning mechanism can generate new control settings if the previous feed forward control settings are outside of a tolerable range via:

DAC_{SETTING},=(V_{FF}*f(Zₚₐₜᵢₑₙₜ)*hₗₑₐᵣₙ(Zₚₐₜᵢₑₙₜ))*m+(b*g(Zₚₐₜᵢₑₙₜ)*qₗₑₐᵣₙ(Zₚₐₜᵢₑₙₜ)) in which hₗₑₐᵣₙ(Zₚₐₜᵢₑₙₜ) and qₗₑₐᵣₙ(Zₚₐₜᵢₑₙₜ) are dynamic learning functions.

In an alternative embodiment of method 300, the impedance of the patient circuit is determined at 302, but the impedance is not measured with the measurement circuit 210 and applied to the test signal 232. Rather, the impedance is determined at 302 from data 152 stored on a memory device 150 of the electrosurgical device 104. In this embodiment, the impedance is associated with the electrosurgical device, and the feedforward control setting is also associated with the electrosurgical device 104. In one embodiment, the feedforward control setting used to determine a puncture signal at 304 is based on previous control settings used in previous application of the puncture signal with previous electrosurgical surgical device of the same type with the electrosurgical generator. In one embodiment, the feedforward control setting is determined from an average of the previous control settings used in previous applications of the puncture signal with previous electrosurgical surgical device of the same type, such as the thirty most recent applications using the electrosurgical generator.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A radiofrequency generator for use in a tissue puncture system for puncturing a tissue in a body, the radiofrequency generator comprising:
a radiofrequency (RF) energy source having an active connector and a return connector, the active connector configured to be coupled to an active electrode of an RF puncture device and the return connector configured to be coupled to a return electrode, the active electrode and return electrode configured to couple to the body and form a patient circuit; and
a controller coupled to the RF energy source, the controller configured to:
determine an impedance of the patient circuit;
determine a puncture signal based on the impedance of the patient circuit, and
cause the RF energy source to apply the puncture signal to the patient circuit.

2. The radiofrequency generator of claim 1, wherein the controller is configured to cause the RF energy source to generate a test signal, wherein the test signal has a lower power than the puncture signal, and to determine the impedance of the patient circuit with the test signal.

3. The radiofrequency generator of claim 1, wherein the determined impedance includes impedance of the RF puncture device.

4. The radiofrequency generator of claim 1, wherein the determined impedance includes impedance between the active connector and the return connector.

5. The radiofrequency generator of claim 1, wherein the determined impedance includes complex impedance having magnitude and phase angle information.

6. The radiofrequency generator of claim 1, wherein the determined puncture signal is based on a feedforward control setting corresponding with the determined impedance.

7. The radiofrequency generator of claim 6, comprising an RF output circuit coupled to the controller, the RF output circuit to provide the RF energy source, wherein the controller is configured to apply the feedforward control setting to the RF output controller.

8. The radiofrequency generator of claim 6, wherein the determined impedance corresponds with a voltage setting, wherein the controller is configured to apply the impedance and voltage setting to determine whether application of the puncture signal exceeds a threshold power setting.

9. The radiofrequency generator of claim 6, wherein the controller is configured to adjust a nominal feedforward control setting based on the impedance to determine the feedforward control setting.

10. The radiofrequency generator of claim 6, wherein the controller is configured to determine the feedforward control setting with a runtime calculation.

11. The radiofrequency generator of claim 6, wherein the controller configured to determine the feedforward control setting with a lookup table.

12. The radiofrequency generator of claim 6, wherein the controller is configured to determine the feedforward control setting and a voltage setting corresponding with the determined impedance with a lookup table.

13. The radiofrequency generator of claim 12, wherein the controller is configured to measure a voltage of the puncture signal in a steady state and update the lookup table.

14. The radiofrequency generator of claim 13, wherein the steady state is after the puncture signal has punctured tissue.

15. The radiofrequency generator of claim 1, wherein the RF puncture device is an electrosurgical transseptal guidewire.

16. A radiofrequency generator for use in a tissue puncture system for puncturing a tissue in a body, the radiofrequency generator comprising:
a radiofrequency (RF) energy source having an active connector and a return connector, the active connector configured to be coupled to an active electrode of an RF puncture device and the return connector configured to be coupled to a return electrode, the active electrode and return electrode configured to couple to the body and form a patient circuit; and
a controller coupled to the RF energy source, the controller configured to:
cause the RF energy source to generate a test signal;
determine an impedance of the patient circuit from the test signal;
determine a puncture signal based on the impedance of the patient circuit, wherein the puncture signal has a higher power than the test signal, wherein the determined puncture signal is based on a feedforward control setting corresponding with the determined impedance; and
cause the RF energy source to apply the puncture signal to the patient circuit.

17. The radiofrequency generator of claim 16, comprising an RF output circuit coupled to the controller, the RF output circuit to provide the RF energy source, wherein the controller is configured to apply the feedforward control setting to the RF output controller.

18. The radiofrequency generator of claim 17, wherein the controller is configured to determine the feedforward control setting and a voltage setting corresponding with the determined impedance with a lookup table, and wherein the controller is configured to measure a voltage of the puncture signal in a steady state and update the lookup table.

19. A method for use in a radiofrequency (RF) generator in a tissue puncture system, the RF generator including an RF energy source having an active connector and a return connector, the active connector configured to be coupled to an active electrode of an RF puncture device and the return connector configured to be coupled to a return electrode, the active electrode and return electrode configured to couple to a patient body and form a patient circuit, the method comprising:
determining an impedance of the patient circuit;
determining a puncture signal based on the impedance of the patient circuit; and
causing the RF energy source to apply the puncture signal to the patient circuit.

20. The method of claim 19, comprising generating a test signal with the RF energy source, wherein the test signal has a lower power than the puncture signal; and determining the impedance of the patient circuit with the test signal.
